(19) United States
(12) Patent Application Publication
(10) Pub. No.: US 2025/0387647 A1
(43) Pub. Date: Dec. 25, 2025

(54) OPTICAL SURFACE TRACKING FOR RADIOTHERAPY

(71) Applicant: Elekta Limited, Crawley (GB)

(72) Inventors: Maja Nilsson, Crawley (GB); Stefan LANGEMARK, Crawley (GB); Kamil KAMINSKI, Crawley (GB)

(21) Appl. No.: 19/240,608

(22) Filed: Jun. 17, 2025

(30) Foreign Application Priority Data
Jun. 20, 2024 (GB) 2408882.5

(51) Int. Cl. A61N 5/10

(52) U.S. Cl. CPC A61N 5/1075; A61N 2005/1076; A61N 2005/1091

(57) ABSTRACT

A phantom configured for evaluating radiotherapy, a radiotherapy device, a method for evaluating imaging for radiotherapy, and a computer-readable medium are provided. The phantom comprises a first three-dimensional structure configured for detection by a kV imaging system. The phantom further comprises a second three-dimensional structure configured for detection by an optical surface tracking system and spaced apart from the first three-dimensional structure.

### CLAIM FOR PRIORITY

This application claims the benefit of priority of British Application No. 2408882.5, filed Jun. 20, 2024, which is hereby incorporated by reference in its entirety.

### TECHNICAL FIELD

This disclosure relates to imaging for radiotherapy, and in particular to a phantom configured for evaluating imaging for radiotherapy, a radiotherapy device, a method for evaluating imaging for radiotherapy and a computer-readable medium.

### BACKGROUND

Radiotherapy can be described as the use of ionising radiation, such as X-rays, to treat a human or animal body. Radiotherapy can be used to treat tumours within the body of a patient or subject. In such treatments, ionising radiation is used to irradiate, and thus destroy or damage, cells which form part of the tumour.

A radiotherapy device can comprise a gantry to support a beam generation system, or other source of radiation, which is rotatable around a patient. For example, for a linear accelerator (linac) device, the beam generation system may comprise a source of radio frequency energy, a source of electrons, an accelerating waveguide, beam shaping apparatus, etc.

### SUMMARY

In radiotherapy treatment, it is desirable to deliver a prescribed dose of radiation to a target region of a subject and to limit irradiation of other parts of the subject, i.e. of healthy tissue. In view of this, a radiotherapy device may comprise one or more imaging devices for capturing images of the patient before and/or during a radiotherapy treatment, which can be used to make adjustments to machine parameters or patient location. Image-guided radiation therapy (IGRT) can improve the accuracy of radiotherapy treatments through confirming that the internal anatomy of the patient is in the expected locations. Surface-guided radiation therapy (SGRT) can improve the accuracy of radiotherapy treatments through confirming that the surface of the patient is in the expected locations.

Before a radiotherapy treatment is started, the patient may be positioned in a suitable position for the radiotherapy treatment. This is referred to herein as the setup phase. The setup phase may involve positioning particular parts of the patient in particular locations and/or at particular angles, in some cases with use of one or more accessories for assisting the patient in taking up and maintaining a desired posture. Subsequently to the setup phase, the radiotherapy treatment may be delivered to the patient. This is referred to herein as the treatment phase. The treatment phase involves delivering a radiotherapy beam to irradiate and thereby treat one or more target regions in the patient.

SGRT can be used in the setup phase to verify that the patient is in the desired location and posture before the radiotherapy treatment starts, and/or can be used in the treatment phase to verify that the patient remains in the desired location and posture during the radiotherapy treatment. For the setup phase and/or the treatment phase, visual tracking of the surface of the patient disposed on a patient positioning surface of the radiotherapy device may be performed. An optical surface tracking (OST) system may be used to provide images of the subject to enable the SGRT to be performed. In other words, the SGRT may be surface-guided in that it is based on images of the surface of the subject provided by the optical surface tracking system.

The optical surface tracking system may generate images from which it is determined that the subject is in an expected location indicating that a tumour is being irradiated, organs at risk are not being irradiated, and treatment should continue. Conversely, the optical surface tracking system may generate images from which it is determined that the subject has moved to an unexpected location indicating that a tumour is being insufficiently irradiated, that an organ at risk is being irradiated more than is desirable, and that treatment should be adjusted, paused or halted. In view of this, inaccuracy of the optical surface tracking system may lead to inaccurate information about the exact location of the subject or anatomical features thereof during treatment, which may lead to a tumour receiving less radiation than would be desirable or organs at risk receiving more radiation than would be desirable.

Moreover, optical surface tracking systems may in some scenarios comprise one or more imaging devices which may be positioned at different locations in a room housing a radiotherapy device. Installation of such imaging devices and of the radiotherapy device itself may involve some variability in the relative locations and orientations of these. In other words, a coordinate system of the radiotherapy device relative to coordinate systems of the one or more imaging devices may be installation-specific and may not be known to a required degree of accuracy from design documents or calculation alone. This may lead to further uncertainty in the accuracy of surface locations of the subject as provided by the optical surface tracking system.

It would be advantageous to provide more accurate imaging of the surface of a subject and thereby to provide more accurate surface-guided radiotherapy. It would also be advantageous to determine whether an optical surface tracking system is providing accurate locations in order to ensure safer and more efficient treatment. It would also be advantageous to provide more accurate and/or more efficient calibration of an optical surface tracking system.

According to an aspect of the present disclosure, there is provided a phantom configured for evaluating imaging for radiotherapy, the phantom comprising: a first three-dimensional structure configured for detection by a kV imaging system; and a second three-dimensional structure configured for detection by an optical surface tracking system and spaced apart from the first three-dimensional structure.

According to a further aspect of the present disclosure, there is provided a radiotherapy device comprising: a radiation source; a patient positioning surface; a kV imaging system; an optical surface tracking system; and the above-mentioned phantom disposed on the patient positioning surface.

According to a further aspect of the present disclosure, there is provided a method for evaluating imaging for radiotherapy, the method comprising: obtaining spatial measurements of the above-mentioned phantom; generating, using a kV imaging system, a kV image of the phantom; generating, using an optical surface tracking system, an optical image of the phantom; and comparing the spatial measurements to the kV image and the optical image.

According to a further aspect, there is provided a computer-readable medium storing instructions which, when executed by a processor, cause performance of the above-mentioned method.

### BRIEF DESCRIPTION OF THE DRAWINGS

Specific embodiments are now described, by way of example only, with reference to the drawings, in which:

FIG. 1a depicts an example of a radiotherapy device or radiotherapy apparatus according to the present disclosure;

FIG. 1b depicts an example of a radiotherapy device or radiotherapy apparatus according to the present disclosure;

FIG. 2 depicts an example of a radiotherapy device or radiotherapy apparatus according to the present disclosure;

FIGS. 3a-d depict an example of a phantom for use in evaluating imaging for radiotherapy according to the present disclosure;

FIG. 4 depicts an example method for evaluating imaging for radiotherapy according to the present disclosure;

FIG. 5 depicts a block diagram of one implementation of a radiotherapy system according to the present disclosure;

FIG. 6 depicts one implementation of a computer program product according to the present disclosure.

### DETAILED DESCRIPTION

The present disclosure provides a phantom configured for use in evaluating imaging for radiotherapy. The phantom includes a first three-dimensional structure configured for detection by a kV imaging system. A kV imaging system, such as a kV imaging system included in a radiotherapy device, can be used to detect or generate images of the first three-dimensional structure. The phantom includes a second three-dimensional structure configured for detection by the optical surface tracking system. An optical surface tracking system, such as an optical surface tracking system included in or associated with the radiotherapy device, can be used to detect or generate images of the second three-dimensional structure. The second three-dimensional structure is spaced apart from the first three-dimensional structure, i.e. they may be described as distinct/different/separate structures. The phantom may be configured for evaluating one or more imaging systems, i.e. may be configured for evaluating the optical surface tracking system and/or the kV imaging system.

The phantom includes separate structures detectable respectively by a kV imaging system and an optical surface tracking system. The ability to image the phantom using the kV imaging system provides an accurate external measurement tool for determining the location of the first three-dimensional structure. The ability to image the phantom using the optical surface tracking system can provide a location of the second three-dimensional structure. This may in some scenarios be a less accurate, more approximate location that that provided for the first three-dimensional structure by the kV imaging system. Pre-determined measurements comprising locations, relative distances or relative displacements of the first and second three-dimensional structures may be compared to corresponding quantities determined based on the kV image and optical image. This may be used to evaluate the optical surface tracking system against the kV imaging system, which may have a better inherent accuracy or the accuracy of which may be better characterised.

The provision of the first three-dimensional structure detectable by the kV imaging system enables the position of the phantom with respect to a coordinate system of the radiotherapy device to be determined accurately. The additional provision of the second three-dimensional structure detectable by the optical surface tracking system enables determination of the position of imaging devices of the optical surface tracking system relative to the phantom. The combination of a known position of the phantom with respect to the coordinate system of the radiotherapy device and the position of the imaging device(s) with respect to the phantom enables determination of the locations of the imaging device(s) with respect to the coordinate system of the radiotherapy device. This enables calibration of the optical surface tracking system such that measurements provided thereby can be directly related to those provided by other imaging devices of the radiotherapy device and to locations at which radiotherapy will be delivered to parts of the subject. This enables integration of different data sources so as to improve the ability to accurately determine the location of the subject at different times and thereby to more accurately deliver radiotherapy in accordance with a treatment plan.

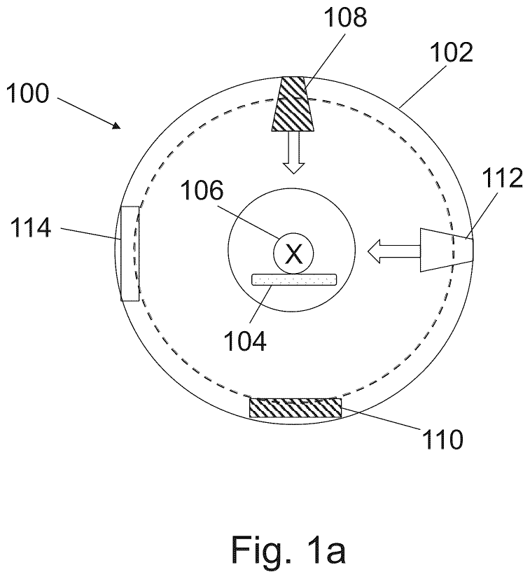

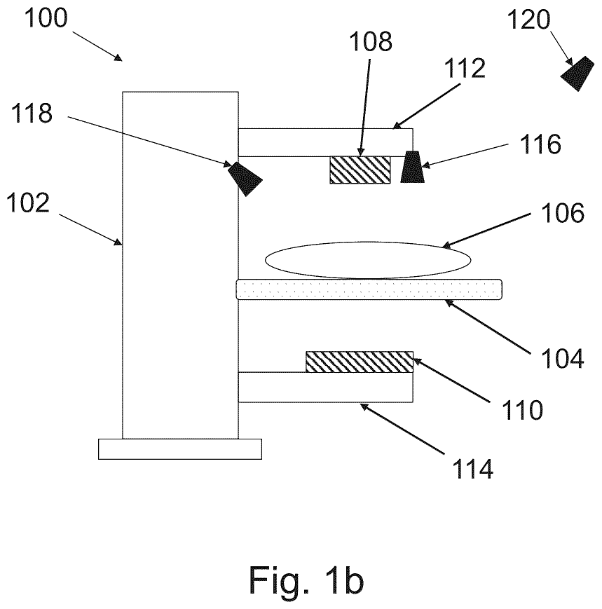

FIG. 1a depicts an example of a radiotherapy device or radiotherapy apparatus 100 according to the present disclosure.

The radiotherapy device 100 depicted in FIG. 1a comprises a rotatable gantry 102 and a patient positioning surface 104 positioned in a treatment volume of the device. The gantry 102 may be ring-shaped. In other words, the gantry 102 may be a ring-gantry. A patient or subject 106 is positioned on the patient positioning surface 104 during radiotherapy treatment. The radiotherapy device 100 may comprise a bore defined by the ring-shaped gantry 102, within which the subject 106 is positioned during treatment. Alternatively, the radiotherapy device may comprise one or more arms connected to and projecting from the front surface of the gantry 102, the arm(s) supporting one or more components of the radiotherapy device 100. The patient positioning surface 104 may be moveable in one or more translational degrees of freedom and one or more rotational degrees of freedom. The patient positioning surface 104 may be used to move the subject 106 from a setup position to a treatment position closer to or encircled by the gantry 102, for example by translating the subject 106 in a direction parallel to the central axis of the gantry 102. The movement of the patient positioning surface 104 may be effected and controlled by one or more actuators and/or motors.

The radiotherapy device 100 comprises one or more sources of kV or MV radiation and one or more detectors configured to detect the kV or MV radiation to generate a plurality of images of a subject between the source and the detector. In particular, the radiotherapy device 100 may comprise a treatment beam source 108 configured to emit or direct therapeutic radiation, e.g. MV energy radiation, towards the subject 106. The treatment beam source 108 may be described as an MV beam source. The treatment beam source 108 may emit radiation suitable for treating a subject 106, which may also be radiation suitable for generating one or more images of the subject 106.

The treatment beam source 108 of the radiotherapy device 100 is configured to deliver a radiation beam towards a radiation isocentre/isocenter (marked with an ‘X’) in FIG. 1a. As depicted in FIG. 1a, the subject 106 is disposed such that the radiation isocentre coincides with the subject 106 or a part of the subject 106, i.e. a part corresponding to an anatomical location of a tumour. The radiation isocentre is substantially located on the axis of rotation at the centre of the gantry 102 regardless of the angle at which the treatment beam source 108 is placed. The isocentre of the kV imaging system may be the same or substantially the same as the radiation isocentre.

The treatment beam source 108 may comprise or have coupled thereto a source of radiofrequency waves, an electron source, a waveguide in which the electrons may be accelerated towards a heavy metal, e.g. tungsten, target to produce high energy photons, and a collimator, such as a multi-leaf collimator, configured to collimate and shape the resulting photons and thus produce a treatment beam. The source of radiofrequency waves may be coupled to the waveguide via a circulator, and may be configured to pulse radiofrequency waves into the waveguide. Radiofrequency waves may pass from the source of radiofrequency waves through an RF input window and into an RF input connecting pipe or tube. The source of electrons, such as an electron gun, may also be coupled to the waveguide and may be configured to inject electrons into the waveguide. In the electron gun, electrons may be thermionically emitted from a cathode filament as the filament is heated. The temperature of the filament controls the number of electrons injected. The injection of electrons into the waveguide may be synchronised with the pumping of the radiofrequency waves into the waveguide. The design and operation of the source of radiofrequency waves, electron source and the waveguide may be such that the radiofrequency waves accelerate the electrons to very high energies as the electrons propagate through the waveguide.

The design of the waveguide depends on whether the linac accelerates the electrons using a standing wave or travelling wave, though the waveguide typically comprises a series of cells or cavities, each cavity connected by a hole or ‘iris’ through which the electron beam may pass. The cavities are coupled in order that a suitable electric field pattern is produced which accelerates electrons propagating through the waveguide. As the electrons are accelerated in the waveguide, the electron beam path may be controlled by a suitable arrangement of steering magnets, or steering coils, which surround the waveguide. The arrangement of steering magnets may comprise, for example, two sets of quadrupole magnets.

Once the electrons have been accelerated, they may pass into a flight tube. The flight tube may be connected to the waveguide by a connecting tube. This connecting tube or connecting structure may be called a drift tube. The electrons travel toward a heavy metal target which may comprise, for example, tungsten. Whilst the electrons travel through the flight tube, an arrangement of focusing magnets act to direct and focus the beam on the target.

To ensure that propagation of the electrons is not impeded as the electron beam travels toward the target, the waveguide may be evacuated using a vacuum system comprising a vacuum pump or an arrangement of vacuum pumps. The pump system is capable of producing ultra-high vacuum (UHV) conditions in the waveguide and in the flight tube. The vacuum system also ensures UHV conditions in the electron gun. Electrons can be accelerated to speeds approaching the speed of light in the evacuated waveguide.

The treatment beam source 108 may comprise a heavy metal target toward which the high energy electrons exiting the waveguide are directed. When the electrons strike the target, X-rays are produced in a variety of directions. A primary collimator may block X-rays travelling in certain directions and pass only forward travelling X-rays to produce a treatment beam. The X-rays may be filtered and may pass through one or more ion chambers for dose measuring. The beam can be shaped in various ways by beam-shaping apparatus, for example by using a multi-leaf collimator, before it passes into the patient as part of radiotherapy treatment.

In some implementations, the treatment beam source 108 is configured to emit either an X-ray beam or an electron particle beam. Such implementations allow the device to provide electron beam therapy, i.e. a type of external beam therapy where electrons, rather than X-rays, are directed toward the target region. It is possible to ‘swap’ between a first mode in which X-rays are emitted and a second mode in which electrons are emitted by adjusting the components of the linac. In essence, it is possible to swap between the first and second mode by moving the heavy metal target in or out of the electron beam path and replacing it with a so-called ‘electron window’. The electron window is substantially transparent to electrons and allows electrons to exit the flight tube.

The radiotherapy device 100 comprises a treatment beam detector or target 110. The treatment beam detector 110 may be described as an MV detector. Once the radiation emitted from the treatment beam source 108 has passed through the patient 106, the radiation continues towards treatment beam detector 110, where it is blocked/absorbed. The treatment beam detector 110 may comprise or include an imaging panel. The treatment beam detector 110 may be configured to produce signals indicative of the intensity of radiation incident on the treatment beam detector 110. In use, these signals are indicative of the intensity of radiation which has passed through the subject 106. These signals may be processed to form an image of the subject 106. This process may be described as the imaging apparatus and/or the treatment beam detector 110 capturing an image. The treatment beam detector 110 may form part of an electronic portal imaging device (EPID). EPIDs are generally known to the skilled person and will not be discussed in detail herein. The treatment beam source 108 and the treatment beam detector 110 may be fixed or attached to the gantry so that they are rotatable with the gantry, i.e. so that they rotate as the gantry rotates.

The radiotherapy device 100 comprises a kV imaging system comprising a kV beam source 112 and a kV detector or target 114. The kV beam source 112 is configured to emit or direct imaging radiation, for example X-rays, towards the subject 106. As the skilled person will appreciate, the kV beam source 112 may be an X-ray tube or other suitable source of X-rays. The kV beam source 112 is configured to produce kV energy radiation. Once the kV radiation has passed from the kV beam source 112 and through the subject 106, the radiation continues towards kV detector 114. The kV detector 114 may comprise or include an imaging panel. The kV detector 114 may be configured to produce signals indicative of the intensity of radiation incident on the kV detector 114. In use, these signals are indicative of the intensity of radiation which has passed through the subject 106. These signals may be processed to form an image of the subject 106. This process may be described as the imaging apparatus and/or the kV detector 114 capturing an image. The kV beam source 112 and the kV detector 114 may be fixed or attached to the gantry so that they are rotatable with the gantry, i.e. so that they rotate as the gantry rotates. By taking images at multiple angles around the subject 106 it is possible to produce a 3D image of the patient, for example using tomographic reconstruction techniques.

In the illustrated example, the treatment beam source 108 and the kV beam source 112 are mounted on the gantry such that a treatment beam emitted from the treatment beam source 108 travels in a direction that is generally perpendicular to that of the imaging beam emitted from the kV beam source 112. Pulsing of radiation from the treatment beam source 108 may be synchronised with reading out of data at the treatment beam detector 110. Pulsing of radiation from the kV beam source 112 may be synchronised with reading out of data at the kV detector 114. Timing signals may be communicated from a controller of the radiotherapy device to one or more of these components in order to provide this synchronisation. The treatment beam detector 110 and/or the kV detector 114 may comprise a flat panel imager. The flat panel imager of the kV detector 114 may be different to the flat panel imager of the treatment beam detector 110 since it is attuned to the different (i.e. lower) energies of the kV radiation.

The flat panel imager may comprise a scintillator. Radiation incident on the scintillator will produce light. The flat panel imager may comprise an array of photodiodes and transistors, each corresponding to a particular pixel of the detector/flat panel imager. The light from the scintillator impinging on the photodiodes creates respective electronic signals which are gated by the respective transistors. These electronic signals are extracted from the flat panel array via read-out electronics to form a digital data stream that is used to construct an image. Generally, the pixel elements of such detectors work by outputting a respective signal in which the total charge passed reflects the total incident radiation since the last time the pixel was read. As radiation is incident on the pixel, it causes ionisation and the resulting charge is retained. When the pixel is enabled, i.e. when it is triggered to release its signal, that charge is output to be counted. The flat panel imager may comprise an interpreter configured to receive the signal outputs. The interpreter may comprise an integrator configured to integrate the signal outputs to measure the charges collected at the respective pixels and thus provide an indication of the radiation received by the pixels of the flat panel imager. This can be used to identify the shape and location of objects (e.g. the subject) between the source and detector through the relative lack of radiation received at the pixels for which the radiation from the source was blocked by the object.

The pixels of the detector may be arranged in a rectilinear manner with the pixels in straight rows and columns. The intersection of a particular row with a particular column therefore defines a specific pixel. Each column may have a common output line which allows the charge that has accumulated on each pixel to escape to the integrator where it is multiplexed with the outputs of other columns. This may enable the entire line of pixels to be read out at the same time. The detector may comprise scanning control electronics which enable each row to be read sequentially, with the whole row read at substantially the same time. The integrator is then reset, and the next row is enabled. Thus, data from the rows of pixels may be read out sequentially until a complete image or frame is obtained, following which the reading out may begin again at the first row.

Because the gantry 102 is rotatable, the treatment beam can be delivered to a patient from a range of angles. Similarly, the patient can be imaged from a range of angles. As the skilled person will appreciate, the gantry 102 can be rotated to any of a number of angular positions relative to a patient. The treatment beam source 108 may direct radiation toward the patient at each or a number of these angular positions, according to a treatment plan. The gantry 102 may be configured to rotate to a number of discrete locations and/or to rotate continuously for a given time period. In other words, the gantry 102 can be rotated by 360 degrees around the subject 106, and in fact can continue to be rotated past 360 degrees. The treatment beam source 108 may be configured to irradiate the subject 106 at the one or more of the discrete locations and/or to continuously irradiate the subject 106 as it is rotated by the gantry 102. The angles from which radiation is applied, and the intensity and shape of the therapeutic beam, may depend on a specific treatment plan pertaining to a given subject 106.

The radiotherapy device 100 additionally comprises a controller (not shown). The controller is a computer, processor, or other processing apparatus. The controller may be formed by several discrete processors; for example, the controller may comprise a processor for each of the various individual components of the radiotherapy device as described herein. The controller is communicatively coupled to a memory, e.g. a computer readable medium. The controller may be communicatively coupled to one, multiple or all of the various individual components of the radiotherapy device as described herein. As used herein, the controller may also be referred to as a control device.

The radiotherapy device and/or the controller may be configured to perform any of the method steps presently disclosed and may comprise computer executable instructions which, when executed by a processor cause the processor to perform any of the method steps presently disclosed, or when executed by the controller cause the controller to perform any of the method steps presently disclosed, or when executed by the radiotherapy device cause the radiotherapy device to perform any of the method steps presently disclosed. Any of the steps that the radiotherapy device and/or the controller is configured to perform may be considered as method steps of the present disclosure and may be embodied in computer executable instructions for execution by a processor. A computer-readable medium may comprise the above-described computer executable instructions.

The radiotherapy device 100 may be described as or comprise a linac. In some examples, the radiotherapy device 100 may be an MR-linac comprising an MR imaging apparatus configured to generate MR images of the subject 106. The MR imaging apparatus may be configured to obtain images of the subject 106 positioned, i.e. located, on the couch 104. The MR imaging apparatus may also be referred to as an MR imager. The MR imaging apparatus may be a conventional MR imaging apparatus operating in a known manner to obtain MR data, for example MR images. The skilled person will appreciate that such a MR imaging apparatus may comprise a primary magnet, one or more gradient coils, one or more receive coils, and an RF pulse applicator. The operation of the MR imaging apparatus is controlled by the controller. Alternatively or in addition to MR imaging, one or more other imaging techniques, modalities, sensors or detectors may be used, such as CT/X-ray, PET, optical imaging/cameras, infra-red imaging, ultra-sound imaging or time-of-flight techniques. Any one or more of these may be used before or during treatment of a subject 106.

The radiotherapy device 100 also comprises several other components and systems as will be understood by the skilled person. For example, in order to ensure the linac does not leak radiation, appropriate shielding may also be provided.

FIG. 1b depicts a further example of a radiotherapy device or radiotherapy apparatus according to the present disclosure. The features of FIG. 1b may be the same as or correspond to features of FIG. 1a or be combinable with the features of FIG. 1a. While FIG. 1a depicts a front view of the radiotherapy device 100, FIG. 1b depicts a side view of the radiotherapy device 100.

FIG. 1b depicts the gantry 102, the patient positioning surface 104, the subject 106, the treatment beam source 108 and the treatment beam detector 110 corresponding to those depicted in FIG. 1a. In FIG. 1b, the radiotherapy device 100 is a C-arm radiotherapy device. In FIG. 1b, the treatment beam source 108 and the treatment beam detector 110 are projected longitudinally from the gantry 102. In particular, the treatment beam source 108 may be disposed on a support arm 112, which may connect the radiation source 100 to the gantry 102. The treatment beam detector 110 may be disposed on a support arm 114, which may connect the treatment beam detector 110 to the gantry 102.

FIG. 1b depicts a plurality of imaging devices 116, 118, 120 of the radiotherapy device 100. A first imaging device 116 may be fixed to the support arm 112. A second imaging device 118 may be fixed to the gantry 102. A third imaging device 120 may be fixed to a wall or ceiling of the treatment room or to a free-standing support. The positions of these imaging devices 116, 118, 120 are provided merely by way of non-limiting example. One or more additional imaging devices may be present in corresponding or additional locations. One or more of the depicted imaging devices 116, 118, 120 may not be present in some examples.

The radiotherapy device 100 may be said to comprise the plurality of imaging devices 116, 118, 120 or may be said to be coupled to (e.g. communicatively coupled to) the plurality of imaging devices. The plurality of imaging devices may be said to be associated with or disposed around or facing or viewing the radiotherapy device 100, or to be configured to be arranged in this manner. As used herein, an imaging device may also be referred to as a sensor or a detector or a camera and may be configured to generate optical images of the radiotherapy device 100, of the subject 106 and/or of a phantom as described herein. The plurality of imaging devices 116, 118, 120 may be configured to monitor the position or location of the subject 106, or a part or surface thereof. The plurality of imaging devices 116, 118, 120 may constitute or be comprised in an optical surface tracking system, which may be configured to enable or facilitate surface-guided radiotherapy. The optical surface tracking system may comprise one or more of these imaging devices 116, 118, 120. The optical surface tracking system may also comprise a controller, and/or may be coupled to a controller of the radiotherapy device.

The imaging devices 116, 118, 120 of the optical surface tracking system may be configured to use any suitable imaging modality. The imaging devices 116, 118, 120 may comprise 2D cameras/technologies and/or 3D cameras/technologies. The imaging devices may comprise one or more visible light cameras (e.g. one or more 2D RGB cameras), one or more structured light cameras, one or more LIDAR cameras, one or more stereo vision cameras and/or one or more time-of-flight cameras. The optical surface tracking system may be configured to monitor the respective locations of a plurality of points on the surface of the subject 106 and determine displacements of each of the points from their previous positions. The optical surface tracking system may be configured to monitor the surface of the subject 106 and its displacement relative to a pre-treatment scan. The optical surface tracking system may be used to infer the locations and/or motions of the internal anatomy of the subject 106.

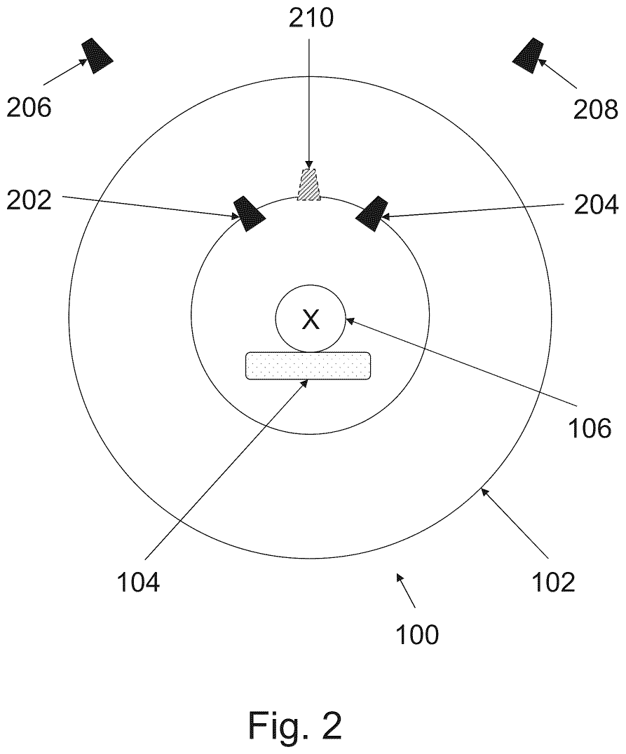

FIG. 2 depicts a further example of a radiotherapy device or radiotherapy apparatus according to the present disclosure. FIG. 2 depicts the radiotherapy device 100 from its front side, i.e. from a perspective facing the front of the rotatable gantry 102. The features of FIG. 2 are combinable with the features of FIG. 1a. In other words, the radiotherapy device 100 depicted in FIG. 2 may be the same radiotherapy device 100 depicted in FIG. 1a, with corresponding features, but FIG. 2 emphasises different features of the radiotherapy device 100 relative to FIG. 1a for ease of illustration.

The radiotherapy device depicted in FIG. 2 comprises the gantry 102 and the patient positioning surface 104. The subject 106 is disposed on the patient positioning surface 104, for example within a bore of the gantry 102/of the radiotherapy device 100. While FIG. 1b depicts the radiotherapy device 100 with a C-arm configuration, FIG. 2 depicts the radiotherapy device 100 with a bore-based configuration. The features and techniques of the present disclosure are applicable to both C-arm and bore-based radiotherapy devices 100 without limitation.

FIG. 2 depicts a plurality of imaging devices 202, 204, 206, 208, 210 of the radiotherapy device 100. One or more of these imaging devices 202, 204, 206, 208, 210 may correspond to or be similar to the imaging devices 116, 118, 110 described in relation to FIG. 1b, such that features described in relation to the imaging devices 202, 204, 206, 208, 210 may also be applicable to imaging devices 116, 118, 110 and vice versa. The radiotherapy device 100 may be said to comprise the plurality of imaging devices or may be said to be coupled to (e.g. communicatively coupled to) the plurality of imaging devices. The plurality of imaging devices may be said to be associated with or disposed around or facing or viewing the radiotherapy device 100, or to be configured to be arranged in this manner. As used herein, an imaging device may also be referred to as a sensor or a detector or a camera and may be configured to generate optical images of the radiotherapy device 100, of the subject 106 and/or of a phantom as described herein. The plurality of imaging devices may be configured to monitor the position or location of the subject 106, or a part or surface thereof. The plurality of imaging devices 202, 204, 206, 208, 210 may constitute or be comprised in an optical surface tracking system, which may be configured to enable or facilitate surface-guided radiotherapy. The optical surface tracking system may comprise one or more of these imaging devices 202, 204, 206, 208, 210. The optical surface tracking system may also comprise a controller, and/or may be coupled to a controller of the radiotherapy device.

The imaging devices 202, 204, 206, 208, 210 of the optical surface tracking system may be configured to use any suitable imaging modality. The imaging devices 202, 204, 206, 208, 210 may comprise 2D cameras/technologies and/or 3D cameras/technologies. The imaging devices may comprise one or more visible light cameras (e.g. one or more 2D RGB cameras), one or more structured light cameras, one or more LIDAR cameras, one or more stereo vision cameras and/or one or more time-of-flight cameras. The optical surface tracking system may be configured to monitor the respective locations of a plurality of points on the surface of the subject 106 and determine displacements of each of the points from their previous positions. The optical surface tracking system may be configured to monitor the surface of the subject 106 and its displacement relative to a pre-treatment scan. The optical surface tracking system may be used to infer the locations and/or motions of the internal anatomy of the subject 106.

The rotatable gantry 102 may comprise a front, a rear, and a bore within the gantry 102 and oriented between the front and the rear. The front corresponds to the side of the rotatable gantry 102 which the patient support surface 104 and/or the subject 106 enter and exit the bore from. The main base of the patient support surface 104 is located to the side of the rotatable gantry 102 corresponding to the front. This is also the side where patient setup is performed. The rear is the opposite surface of the rotatable gantry 102 to the front, with the bore therebetween. The interior surface of the bore may be perpendicular to the front and the rear and may connect the front to the rear.

The optical surface tracking system may comprise any number of (i.e. one or more) imaging devices. FIG. 2 depicts by way of non-limiting example some possible locations of such imaging devices. In other examples, a single imaging device or multiple imaging devices may be provided attached to the front of the gantry 102 or to an outer housing at the front of the radiotherapy device 100.

As depicted in FIG. 2, the optical surface tracking system may comprise one or more near-bore imaging devices 202, 204. The one or more near-bore imaging devices 202, 204 may be located on, at or close to a front surface of the rotatable gantry 102. The one or more near-bore imaging devices 202, 204 may be located at a transition point or in a transition region or in a curved region or at an angle between the front and the interior of the bore of the radiotherapy device 100. The one or more near bore imaging devices 202, 204 may be attached to a front side of the radiotherapy device 100. In some examples, the one or more near-bore imaging devices 202, 204 may be located inside the bore proximal to the front of the rotatable gantry 102. The one or more near-bore imaging devices 202, 204 may be arranged to be at a vertical height above the vertical height of the subject 106 on the patient support surface 104. The one or more near-bore imaging devices 202, 204 may disposed at a vertical height above the vertical height of the axis of rotation of the radiotherapy device 100. The one or more near-bore imaging devices 202, 204 may be disposed at a vertical height between the vertical height of the patient support surface 104 and the vertical height of the top of the bore, or at the same vertical height as the top of the bore. The one or more near-bore imaging devices 202, 204 may be disposed, for example, in a top half, third, quarter, fifth, tenth, or twentieth of the vertical extent of the bore.

FIG. 2 depicts a first near-bore imaging device 202 and a second near-bore imaging device 204. The first near-bore imaging device 202 and the second near-bore imaging device 204 may be located at the same vertical height. The first near-bore imaging device 202 and the second near-bore imaging device 204 may be located horizontally adjacent to each other, i.e. with a certain horizontal (left-right) separation. The first near-bore imaging device 202 may be oriented towards the centre/center of the bore along a horizontal direction and the second near-bore imaging device 204 may be oriented towards the centre of the bore along the horizontal direction. In other words, the views of the first near-bore imaging device 202 and the second near-bore imaging device 204 may intersect (in the horizontal direction). This may provide views of the subject 106 from opposing horizontal directions, thereby providing a more complete view of the surfaces of the subject 106.

As depicted in FIG. 2, the optical surface tracking system may comprise one or more setup imaging devices 206, 208. The one or more setup imaging devices 206, 208 may be located close to or adjacent to the front of the rotatable gantry 102. The one or more setup imaging devices 206, 208 may be located on the side of the radiotherapy device 100 corresponding to the front of the rotatable gantry 102, i.e. closer to the front than the rear. The one or more setup imaging devices 206, 208 may be oriented vertically downwards or may be oriented at least partially towards the front of the rotatable gantry 102, e.g. at an angle between vertically downwards and the axis of rotation running from the front to the rear. The one or more setup imaging devices 206, 208 may be configured to view the subject 106 in a setup/pre-treatment position, i.e. with the support surface 104 withdrawn from the bore. The one or more setup imaging devices 206, 208 may be disposed at a vertical height higher than the top of the bore. The one or more setup imaging devices 206, 208 may be disposed at a vertical height level with a part (e.g. a top arc) of the rotatable gantry 102 or higher than the top of the rotatable gantry 102. The one or more setup imaging devices 206, 208 may be oriented to coincide with the centre of the bore/with the isocentre. While these imaging devices are referred to as ‘setup imaging devices’, it will be appreciated that they may be dedicated to setup, but also be used when the patient support surface 104 and the subject 106 are within the bore, e.g. during treatment, such as to image the parts of the surface which are not visible for other imaging devices.

FIG. 2 depicts a first setup imaging device 206 and a second setup imaging device 208. The first setup imaging device 206 and the second setup imaging device 208 may be located at the same vertical height. The first setup imaging device 206 and the second setup imaging device 208 may be located horizontally adjacent to each other, i.e. with a certain horizontal (left-right) separation. The horizontal separation of the first setup imaging device 206 and the second setup imaging device 208 may be greater than the horizontal separation between the first near-bore imaging device 202 and the second near-bore imaging device 204. The first setup imaging device 206 may be oriented towards the centre of the bore along a horizontal direction and the second setup imaging device 208 may be oriented towards the centre of the bore along the horizontal direction. In other words, the views of the first near-bore imaging device 206 and the second near-bore imaging device 208 may intersect (in the horizontal direction). This may provide views of the subject 106 from opposing horizontal directions, thereby providing a more complete view of the surfaces of the subject 106.

As depicted in FIG. 2, the plurality of imaging devices may comprise a rear imaging device 210. The rear imaging device 210 may be positioned outside the bore to the rear of the radiotherapy device 100, i.e. adjacent to the rear of the rotatable gantry 102. The rear imaging device 210 is depicted with dotted lines and shading to indicate that it is located (at least partially) behind the radiotherapy device 100 from the perspective shown in FIG. 2. This is because the perspective shown in FIG. 2 is facing the front of the rotatable gantry 102 and the rear imaging device 210 is adjacent to the rear of the rotatable gantry 102. The rear imaging device 210 may be oriented towards the rear of the rotatable gantry 102/radiotherapy device 100. The rear imaging device 210 may be attached to a rear side of the radiotherapy device 100. In some examples, there may be several, e.g. two, three or four, rear imaging devices 210 positioned adjacent to the rear. In some examples, there may be an array of rear imaging devices 210 positioned adjacent to the rear.

The rear imaging device 210 may be positioned vertically above the centre of the bore, i.e. vertically above the axis of rotation of the rotatable gantry 102. The rear imaging device 210 may be arranged to be at a vertical height above the vertical height of the subject 106 on the patient support surface 104. The rear imaging device 210 may be disposed at a vertical height above the vertical height of the patient support surface 104. The rear imaging device 210 may be disposed at a vertical height level with the vertical height of the top of the bore. Alternatively, the rear imaging device 210 may be disposed at a vertical height below or above the height of the top of the bore. The rear imaging device 210 may be disposed at a vertical height below that of the vertical height of the top of the rotatable gantry 102. For a single rear imaging device 210, the rear imaging device 210 may be horizontally positioned level with the centre of the bore (i.e. in the left-right direction). For multiple rear imaging devices 210, one or more of the rear imaging devices 210 may be positioned either side of the horizontal center of the bore. The one or more rear imaging devices 210 may be oriented to coincide with the centre of the bore/with the isocentre.

While the imaging devices 202, 204, 206, 208, 210 have been described above in terms of imaging the subject 106 positioned on the patient support surface 104, it will be appreciated that, in the context of the present disclosure, these imaging devices 202, 204, 206, 208, 210 may view a phantom as described herein, which is disposed on the patient positioning surface 104, in a corresponding manner.

In some examples, the coordinate systems of the different imaging devices may be mapped onto each other. Locations inside the bore and locations outside the bore may be mapped onto the same coordinate system. The coordinate system of the radiotherapy device 100 or of the room within which it is housed may be used as the reference coordinate system and the respective coordinate systems of each of the plurality of imaging devices may be mapped onto that reference coordinate system. The coordinate systems of one of the plurality of imaging devices may be used as the reference coordinate system and the data and coordinate systems of the other imaging devices of the plurality of imaging devices may be mapped onto that reference coordinate system. The data obtained by each of the plurality of imaging devices may be calibrated as described herein in order to indicate the location of the surface of the subject 106 in the reference coordinate system.

The arrangement of the plurality of imaging devices depicted in FIG. 2 is provided as an example. In some examples, one or more of the plurality of imaging devices may be absent or may have a different location and/or orientation relative to that depicted. In some examples, one or more additional imaging devices may be included in the optical surface tracking system.

One or more of the plurality of imaging devices may be configured to be fixed or supported in various ways (e.g. using mounting means), for example fixed to the ceiling of the room containing the radiotherapy device 100, fixed to a free-standing support, fixed to the rotatable gantry 102 or fixed to an additional gantry or rig of the radiotherapy device 100. The one or more near-bore imaging devices 202, 204 may be fixed to a front side of the radiotherapy device 100, e.g. to an additional gantry or rig of the radiotherapy device 100. The one or more setup imaging devices 206, 208 may be fixed to the ceiling of the room containing the radiotherapy device 100. The rear imaging device 210 may be fixed to the radiotherapy device 100 via mounting means, e.g. via one or more rods, arms and/or clamps.

As will be appreciated, the one or more imaging devices may be fixed to the radiotherapy device 100, to features of the room housing the radiotherapy device 100, or to free-standing supports. They may be moveable and/or pivotable. As such, the locations and orientations of the one or more imaging devices relative to coordinates of the radiotherapy device 100 are not necessarily known, or at least not to a required level of accuracy, following installation. Calibration of the one or more imaging devices can be used to improve the accuracy with which their positions and orientations are known, and thereby increase the accuracy of locations determined based on images they generate.

SGRT can improve the accuracy of radiotherapy treatment by verifying that the surface of the subject is in a position which reflects their position during a pre-treatment scan (e.g. a CT simulation). Since the pre-treatment scan data is what is used to determine how the dose will be deposited in the subject, verifying that the subject is in a corresponding position during treatment helps ensure that dose is applied in the intended anatomical locations. However, if the optical surface tracking system itself is not calibrated or otherwise provides inaccurate measurements of surfaces of the subject, the SGRT based on images generated by the optical surface tracking system will itself be inaccurate. The present disclosure describes techniques for evaluating and verifying the accuracy of the optical surface tracking system and thereby for providing more accurate surface-guided radiotherapy/more accurate radiotherapy treatment.

In radiotherapy, commissioning and device acceptance testing (DAT) are required to ensure that an installed radiotherapy device is working as expected. In addition, routine quality assurance (QA) is required to ensure that the device meets the expected tolerances. For conducting such testing, a phantom may be used. A phantom is a substitute for a subject's body or a part of a subject's body. In other words, a beam of radiation may be delivered to a phantom as part of testing to ensure that the beam of radiation can be safely and accurately delivered to a subject, with parameters of the radiotherapy device and the radiation delivery being as expected or intended within given tolerances. Alternatively, or in addition, imaging may be performed with a phantom in the intended location of a subject in order to verify whether the imaging meets accuracy and performance requirements. A phantom as described herein may be considered to be a substitute for or a physical model of a subject's body or a part of a subject's body. The phantom may not be a subject or a patient or a human or animal or a part thereof.

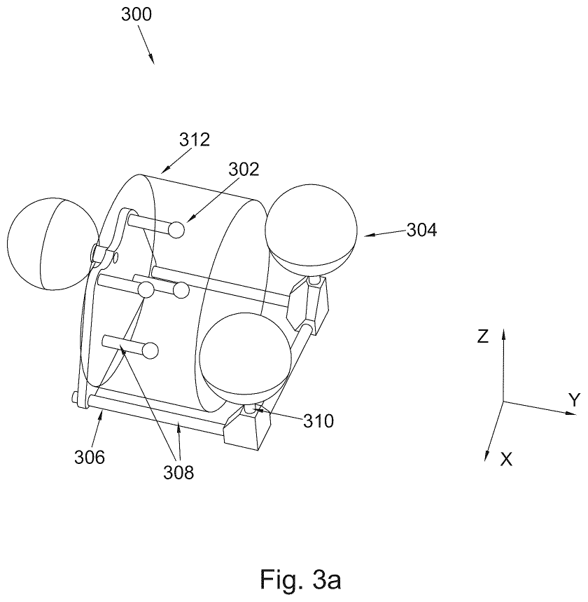

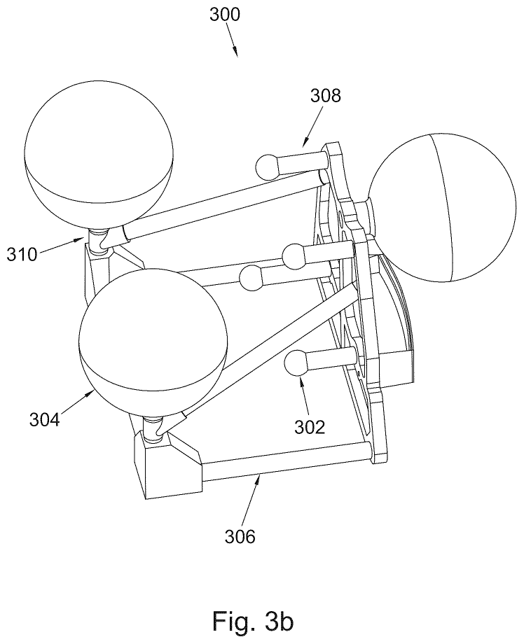

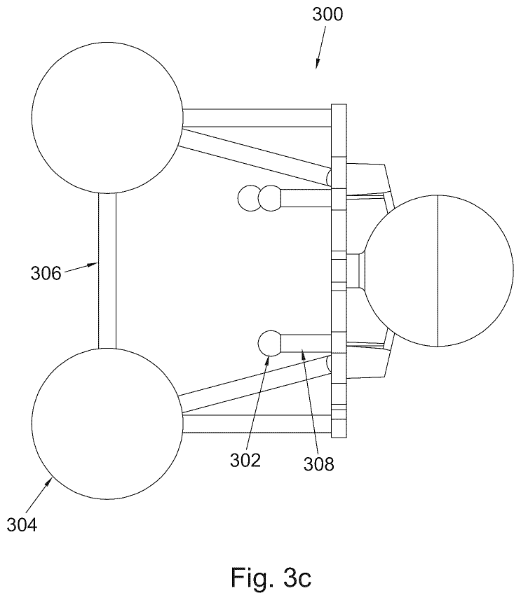

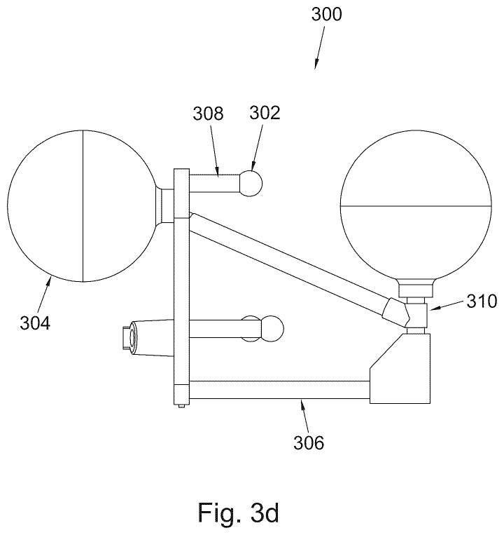

FIGS. 3a-d depict an example of a phantom 300 for use in evaluating imaging for radiotherapy according to the present disclosure. It will be appreciated that the form and structure of the phantom 300 is provided for illustration by way of non-limiting example, and that other forms and structures of phantom are considered as part of the present disclosure.

As depicted in FIG. 3a, the phantom 300 comprises a first three-dimensional structure 302 configured for detection by a kV imaging system. The first three-dimensional structure 302 may be described as detectable or imageable by the kV imaging system, e.g. by the kV beam source 112 and kV detector 114 described in relation to FIG. 1a and FIG. 1b. The first three-dimensional structure 302 may be formed of a radio-opaque material such as a ceramic. X-rays emitted from the kV beam source 112 may not pass through the first three-dimensional structure 302 such that these X-rays may not be incident on pixels of the kV detector 114 aligned with the first three-dimensional structure 302. Since X-rays from the KV beam source 112 are able to pass through other parts of the phantom 300 and the air, these X-rays may be incident on surrounding pixels of the kV detector 114. As such, the location of the first three-dimensional structure 302 can be detected in images generated by the kV imaging system based on the location at which the radiation received is different (i.e. lower).

The first three-dimensional structure 302 may be spherical (as depicted in FIG. 3a), but is not limited thereto. The first three-dimensional structure 302 being spherical in shape may make the mathematical analysis of the imaging data easier or more efficient since a sphere appears spherical/circular in shape from any orientation. Since spheres are relatively easy to form, the first three-dimensional structure 302 being spherical in shape may also simplify manufacturing of the phantom 300. The first three-dimensional structure 302 may be a ceramic ball bearing. In some examples, the first three-dimensional structure 302 may have the shape of a cube, pyramid, prism, etc.

The phantom 300 may comprise a plurality of the above-described first three-dimensional structures 302. The phantom 300 may comprise one, two, three, four, five, six, seven, eight, nine, ten, or more of the first three-dimensional structures 302. Each of the first three-dimensional structures 302 may be spaced apart from each of the other first three-dimensional structures 302. By way of example, the phantom 300 depicted in FIG. 3a comprises four of the first three-dimensional structures 302. This may enable more accurate determination of the location of the phantom 300 since a location of each of the first three-dimensional structures 302 may be detected, which may reduce an overall error margin associated with a determined location of the phantom 300 relative to the kV imaging system. Moreover, providing multiple of the first three-dimensional structures 302 may enable a distance to the phantom 300 and/or an orientation of the phantom 300 to be determined based on distances between the detected locations of the different first three-dimensional structures 302. While having three first three-dimensional structures 302 may be adequate for determining the relevant geometrical relationships, providing four of the first three-dimensional structures 302 provides redundancy and enables additional information regarding measurement error to be obtained.

The phantom comprises a second three-dimensional structure 304 configured for detection by an optical surface tracking system. The second three-dimensional structure 304 may be described as detectable or imageable by the optical surface tracking system, e.g. by the imaging device(s) described above in relation to FIG. 1b and FIG. 2. The second three-dimensional structure 304 may be opaque to visible light. A colour or brightness/darkness of the second three-dimensional structure 304 may be different to that of other parts of the phantom 300. For example, the second three-dimensional structure 304, or at least the outer surface thereof, may be light grey in colour, while some or all of the other parts of the phantom 300, or at least the outer surfaces thereof, may be black in colour. The different colour of the second three-dimensional structure 304 relative to other parts of the phantom 300, and to the room in which the radiotherapy device 100 is housed, may enable the location of the second three-dimensional structure to be identified in images generated by the optical surface tracking system.

The second three-dimensional structure 304 may be spherical (as depicted in FIG. 3a), but is not limited thereto. In a similar manner to the explanation provided in relation to the first three-dimensional structure 302, the spherical shape of the second three-dimensional structure 304 may be beneficial for analysis of image data and for manufacturing. In some examples, the second three-dimensional structure 304 may have the shape of a cube, pyramid, prism, etc.

A width or volume of the second three-dimensional structure 304 may be larger than a width or volume of the first three-dimensional structure 302. This may enable detection of the locations of the second three-dimensional structures 304 with increased accuracy. The optical surface tracking system may generally be less accurate than the kV imaging system such that imaging larger objects using the optical surface tracking system may increase the accuracy of determinations of locations of such objects from generated optical images.

In practice, the field of view and resolution of the kV imaging system may be substantially different to those of the optical surface tracking system. The optical surface tracking system may require the second three-dimensional structures 304 to be positioned with a relatively large distance between them, such that they may not fit within the field of view of the kV imaging system. As such, the first three-dimensional structures 302 may be separated from the second three-dimensional structures 304, e.g. with the first three-dimensional structures 302 positioned between different ones of the second three-dimensional structures 304 as depicted in FIG. 3a, in order to enable accurate imaging of the structures 302, 304 without increasing field of view requirements of the imaging systems.

The phantom 300 may comprise a plurality of the above-described second three-dimensional structures 304. The phantom 300 may comprise one, two, three, four, five, six, seven, eight, nine, ten, or more of the second three-dimensional structures 304. Each of the second three-dimensional structures 304 may be spaced apart from each of the other second three-dimensional structures 304. By way of example, the phantom 300 depicted in FIG. 3a comprises three of the second three-dimensional structures 304. This may enable more accurate determination of the location of the phantom 300 since a location of each of the second three-dimensional structures 304 may be detected, which may reduce an overall error margin associated with a determined location of the phantom 300 relative to the optical surface tracking system. Moreover, providing multiple of the second three-dimensional structures 304 may enable a distance to the phantom 300 and/or an orientation of the phantom 300 to be determined based on distances between the detected locations of the different second three-dimensional structures 304. Providing three of the second three-dimensional structures 304 may enable determination of the location and orientation of the phantom 300 in six degrees of freedom, without need to move the phantom 300. Providing three, and not more, of the second three-dimensional structures 304 may prevent different ones of the second three-dimensional structures 304 from occluding each other from different angles, which may otherwise prevent one or more imaging devices from seeing a full projection of all the second three-dimensional structures 304.

A first subset of the surface the second three-dimensional structure 304 may be visually distinct from a second subset of the surface of the second three-dimensional structure 304. For example, each of the first subset and the second subset may comprise a respective surface corresponding to a respective hemisphere of the second three-dimensional structure 304. The first subset may be a different colour or brightness/darkness relative to the second subset. In some examples, the second three-dimensional structure 304 may comprise a visual marking such as a line provided around a circumference of the second three-dimensional structure 304. Where there are multiple of the second three-dimensional structures 304, the above-described differences in colouring/brightness/darkness may be oriented differently for different ones of the second three dimensional structures 304. Where there are multiple of the second three-dimensional structures 304, an orientation of the above-described visual marking may be different for different ones of the second three dimensional structures 304.

The provision of the above-described colourings/brightnesses/darknesses/markings may enable more accurate identification of the orientation or angle of the phantom 300 relative to the optical surface tracking system.

In some examples, each of the second three-dimensional structures 304 may be uniform in colour. The second three-dimensional structures 304 may be grey in colour, with the other components of the phantom 300 being black in colour. The grey colour of the second three-dimensional structures 304 may be beneficial for accurate imaging of these structures by the optical surface tracking system. The black colour of the rest of the components of the phantom 300 may prevent distortion of the images generated using the optical surface tracking system through reflections from these other components.

In some examples, each second three-dimensional structure 304 may be manufactured by forming two hemispheres with the material inside each hemisphere being milled out. The two hemispheres may be joined together to form the spherical shape depicted in FIG. 3a. This manufacturing approach may advantageously reduce the mass of the phantom 300.

The phantom 300 may comprise a frame 306. As used herein, the frame 306 may also be referred to as a chassis or a support. The frame 306 may comprise one or more panels and/or one or more bars fixedly connected together. The bars may comprise carbon fibre tubes. The first three-dimensional structure(s) 302 and the second three-dimensional structure(s) 304 may be fixedly connected to the frame 306. The first three-dimensional structure(s) 302 and the second three-dimensional structure(s) 304 may be fixedly connected to each other via the frame 306. This may enable the displacement between each of the first three-dimensional structure(s) 302 and the second three-dimensional structure(s) 304 to be rigidly fixed such that it does not vary. This enables accurate determinations to be made regarding the relative locations of these structures when their locations are identified by the kV imaging system and the optical surface tracking system.

The frame 306 may also provide stability to the phantom 300 such that it can be disposed securely on a patient positioning surface 104 of a radiotherapy device 100 (see FIG. 1a, FIG. 1b and FIG. 2) without undesired movement of the phantom 300. The frame 306 and the patient positioning surface 104 may comprise one or more cooperating features for fixedly connecting the phantom 300 to the patient positioning surface 104 in an accurate, pre-determined manner. The phantom may be disposed, in the orientation depicted in FIG. 3a, such that the Y-direction coincides with a central axis of the gantry 102/of the bore of the radiotherapy device 100 (into/out of the page as depicted in FIG. 1a and FIG. 2). The X-direction may correspond to a left-right direction as depicted in FIG. 1a and FIG. 2. The Z-direction may correspond to an up-down direction as depicted in FIG. 1a and FIG. 2.

The first three-dimensional structure 302 may be fixedly connected to the frame 306 via a support column 308. The support column 308 may be formed of a rigid material such as a carbon fibre tube. Each of the first three-dimensional structures 302 may be fixedly connected to the frame 306 via a respective one of the support columns 308.

The second three-dimensional structure 304 may be fixedly connected to the frame 306 via a support column 310. The support column 310 may be formed of a rigid material such as a carbon fibre tube. Each of the second three-dimensional structures 304 may be fixedly connected to the frame 306 via a respective one of the support columns 310. Alternatively, one or more of the second three-dimensional structures 304 may be directly connected to a component of the frame 306, such as a bar comprised in the frame 306.

As depicted in FIG. 3a, the first three-dimensional structure 302 is spaced apart from the second three-dimensional structure 304. As depicted in FIG. 3a, each of the first three-dimensional structures 302 is spaced apart from each of the second three-dimensional structures 304. In other words, these structures may not be physically touching. There may be a separation distance between these structures. There may be free space between these structures.

In use, the phantom 300 may be disposed at the isocentre of the radiotherapy device 100 on the patient positioning surface 114. In some examples, the above-described cooperating features may be used to accurately dispose the phantom 300 at the isocentre. Alternatively, or in addition, this may be enabled by visual markings provided on the phantom for aligning with lasers in the room housing the radiotherapy device 100.

The kV imaging system comprising the kV beam source 112 and the kV detector 114 may be configured to generate images within a kV imaging volume 312 coinciding with, i.e. comprising, the isocentre. The phantom 300 may be structured such that, in use, the one or more first three-dimensional structures 302 are disposed within the kV imaging volume 312. The phantom 300 may be structured such that, in use, the one or more second three-dimensional structures 304 are disposed outside the kV imaging volume 312. This may be enabled, for example, by the first three-dimensional structures 302 being spaced apart from the second three-dimensional structures 304. It may be enabled, for example, by the first three-dimensional structures 302 being grouped together such that a separation distance between them is a certain fraction of the overall width or dimensions of the phantom 300. It may be enabled, for example, by disposing one or more of the second three-dimensional structures 304 on a first side of the first three-dimensional structures 302, and others (e.g. a remainder) of the second three-dimensional structures 304 on a second, opposite side of the first three-dimensional structures 302.

In addition to the above-mentioned considerations regarding the different fields of view and resolutions of the kV imaging system and the optical surface tracking system, providing the first three-dimensional structures 302 spaced apart from the second three-dimensional structures 304 may improve the imaging of these structures by these different imaging systems. For example, this arrangement can prevent issues relating to the second three-dimensional structures 304 being imaged by the kV imaging system, which may otherwise reduce accuracy through disturbing the imaging of and determination of the location of the first three-dimensional structures 302.

As used herein, references to a location of a feature (such as a phantom 300, a first three-dimensional structure 302 and/or a second three-dimensional structure 304) may be used interchangeably with references to a position of that feature. This location or position may be used to refer to a three-dimensional location or position defined along three translational axes, or may be used to refer to a six-dimensional location or position defined along these three translational axes and according to rotation about these three translational axes. In other words, this location or position may be considered to have three degrees of freedom or six degrees of freedom.

FIGS. 3b-d depict additional views of the phantom 300 of FIG. 3a. FIGS. 3b-d depict the first three-dimensional structures 302, the second three-dimensional structures 304, the frame 306, the support column 308 and the support column 310 depicted in FIG. 3a. In FIGS. 3b-d, the kV imaging volume 312 is not depicted for ease of illustration.

As depicted in FIGS. 3b-d, one of the first three-dimensional structures 302 may be displaced from a plane in which the others of the first three-dimensional structures 302 are located. For example, the support column 308 supporting this first three-dimensional structure 302 may be longer than the support columns 308 supporting the other first three-dimensional structures 302. With reference to the coordinate system depicted in FIG. 3a, the first three-dimensional structure 302 may be displaced in the Y-direction relative to an X-Z plane in which the others of the first three-dimensional structures 302 are located. In some examples, one or more of the first three-dimensional structures 302 may be displaced relative to others of the first three-dimensional structures, e.g. displaced by different amounts. Such displacement or displacements can enable the orientation of the phantom 300 to be more accurately determined since it reduces the symmetry of the phantom 300 in different orientations.

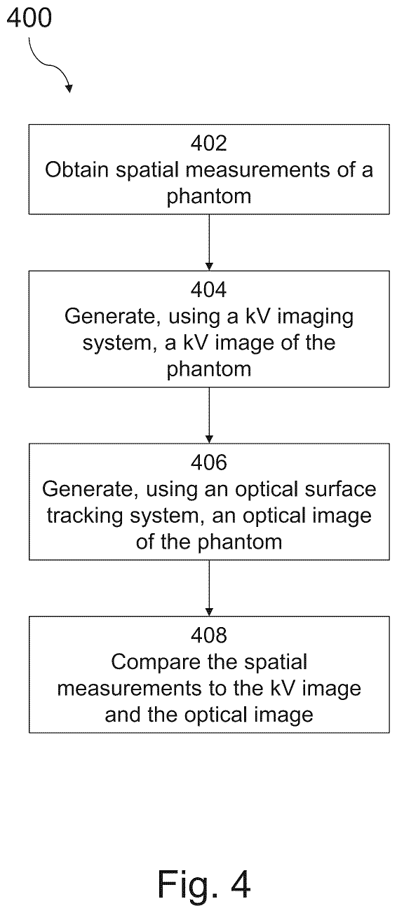

FIG. 4 depicts a method for evaluating imaging for radiotherapy according to the present disclosure. The method may be performed using any of the phantoms described herein, including that referred to in relation to FIGS. 3a-d. The method may be performed using the radiotherapy device 100 described herein, using the kV imaging system described herein, and/or using the optical surface tracking system described herein.

In a step 402, the method comprises obtaining spatial measurements of a phantom 300. In some examples, the spatial measurements may be determined using on or more measuring tools or devices. In some examples, the spatial measurements may be determined from data sheets characterising the dimensions/geometry of the phantom. In some examples, based on the spatial measurements, a respective centre point of each of the first three dimensional structures 302 and the second three dimensional structures 304 may be determined. The center points may be determined by measuring the phantom 300 and all of the structures 302, 304 thereof in relation to each other. For example, when each of these structures is spherical, a respective centre point of each of the spheres may be determined based on the spatial measurements. Respective centre points of the structures may be determined based on obtained spatial measurements for points on the surfaces of each of the structures. For example, a centre of a sphere may be determined by taking an average of two locations at opposite points on the surface of the sphere. The centre points may be determined using a model of the phantom 300, and may thereafter be stored in the model.

In some examples, the second three-dimensional structure 304 may be formed from two hemispheres, e.g. glued together with epoxy. In some examples, standard carbon fibre tubes may be used as the support columns 308 and/or the support columns 310. To simplify the manufacturing process, strict tolerances may not be applied on these or other components of the phantom 300. Instead, once the phantom 300 is formed, it may be measured in order to get exact coordinates of the respective structures. The first three-dimensional structures 302 may be bearing balls, and may have a very exact shape. The second three-dimensional structures may be machined, and have a less exact shape (e.g. a less exact roundness). While manufacturing of calibration objects often aims at producing such calibration objects with very low tolerances, the present disclosure recognises that this may be difficult and inefficient in practice. Instead, the techniques of the present disclosure accept that the locations/shapes of different components of the phantom 300 may have larger tolerances than would be desirable for calibrating the imaging systems, and addresses this through obtaining real spatial measurements of the phantom 300 to compare to the generated images. This may improve the accuracy and efficiency of evaluation/calibration of the imaging systems described herein.

In some examples, a measuring arm may be used to determine the spatial measurements. Such a measuring arm may have a number of articulated sections, with the location of a distal end of the measuring arm being determinable based on the known lengths of each of the articulated sections and the sensed angles between them at a given time. The distal end of the measuring arm may comprise a probe for contacting surfaces of the phantom 300 or a laser scanner for measuring a distance to and shape of the phantom 300. The spatial measurements of the phantom 300 may be determined using the data provided by the contact probe/laser scanner in combination with the determined location of the distal end of the measuring arm.

In a step 404, the method comprises generating, using a kV imaging system, a kV image of the phantom 300. Since each of the first three-dimensional structures 302 is configured for detection by the kV imaging system, the kV image may depict each of the first three-dimensional structures 302, i.e. may indicate their respective locations within the kV image. The generating of the kV image may be performed with the phantom disposed on the patient positioning surface 104 of the radiotherapy device 100.

In a step 406, the method comprises generating, using an optical surface tracking system, an optical image of the phantom 300. Since each of the second three-dimensional structures 304 is configured for detection by the optical surface tracking system, the optical image may depict each of the second three-dimensional structures 304, i.e. may indicate their respective locations within the optical image. The generating of the optical image may be performed with the phantom disposed on the patient positioning surface 104 of the radiotherapy device 100.

In a step 408, the method comprises comparing the spatial measurements to the kV image and the optical image. This may comprise comparing the above-described model of the phantom 300 to the kV image and the optical image. A location of each of the first dimensional structures 302 in the spatial measurements/model may be compared to a location of the respective first three-dimensional structures 302 in the kV image. A location of each of the second three-dimensional structures 304 in the spatial measurements/model may be compared to a location of the respective second three-dimensional structures 304 in the optical image. A separation between each pair of the first three-dimensional structures 302 and the second three-dimensional structures 304 in the spatial measurements/model may be compared to a separation between a respective pair of the first three-dimensional structures 302 and the second three-dimensional structures 304 determined based on the kV image and the optical image.

Since the spatial measurements between the first and second three-dimensional structures 302, 304 is known from step 402 described above, it can be determined to a high degree of accuracy where the second three-dimensional structures 304 are by determining the location of the first three-dimensional structures 302 using the kV image(s) generated in step 404. For example, this location may be with respect to the isocentre, assuming the kV imaging system is calibrated with the rest of the machine. The images of the second three-dimensional structures 304 generated in step 406 can then be used with these known locations of the second three-dimensional structures 304 in order to calibrate the optical surface tracking system, for example by accurately determining the locations and error margins of the imaging devices of the optical surface tracking system. This may comprise determining rigid transforms (in six degrees of freedom) based on the three-dimensional centres of the structures 302 and/or the structures 304. Alternatively, or in addition, differences in size/scale of the structures 302 and/or the structures 304 may be used for these determinations.

The spatial measurements/model may provide a ground truth for the phantom 300. In other words, the locations of the first three-dimensional structures 302 and the second three-dimensional structures 304 may be measured very accurately such that it may be assumed that an error in their locations and separations is negligible. The kV imaging system may provide highly accurate locations of the first three-dimensional structures 302 in the kV images. The kV imaging system may be calibrated and well characterised and inherently capable of highly accurate spatial measurements. The kV imaging system may be incorporated into the radiotherapy device 100 such that it may provide locations of the first three-dimensional structures 302 in the coordinate system of the radiotherapy device 100.

However, the spatial accuracy of the optical surface tracking system may not inherently be as accurate as would be desired. The spatial accuracy may depend on the resolution/pixel density of the imaging devices of the optical surface tracking system and their separation from the phantom 300. Since imaging devices of the optical surface tracking system may not necessarily be incorporated into the body of the radiotherapy device 100 itself, their locations and orientations with respect to the coordinate system of the radiotherapy device may not known a priori. Therefore, the comparing of the spatial measurements to the kV image and the optical image may be used to determine an error margin for the optical surface tracking system, i.e. for locations of the second three-dimensional structures 304 in optical images generated by the optical surface tracking system. This may be based on the ground truth locations provided by the spatial measurements and a known, small, error margin for the kV imaging system.

The method may comprise moving the patient positioning surface 104 with the phantom 300 disposed thereon to an adjusted location and/or an adjusted orientation. A second kV image of the phantom 300, i.e. of the first three-dimensional structures 302, may be generated using the kV imaging system. A second optical image of the phantom 300, i.e. of the second three-dimensional structures 304, may be generated using the optical surface tracking system. The spatial measurements may be compared to the second kV image and to the second optical image in a similar manner to that described above in relation to step 408. This may enable increased accuracy in the characterisation/calibration or may enable characterisation/calibration of further imaging devices of the optical surface tracking system.

The phantom 300 and the proposed techniques described herein may be used for calibrating an optical surface tracking system, i.e. for calibrating the locations and orientations of the imaging devices with respect to a coordinate system of the radiotherapy device 100. The calibration may based on the phantom 300 being in a known location with respect to the coordinate system of the radiotherapy device 100. This may be determined by disposing the phantom 300 in a specific location with respect to the radiotherapy device 100, for example at the isocentre. Alternatively, or in addition, an additional imaging modality may be used to determine this location, for example the kV imaging system. This may provide further accuracy and/or may enable determining of the position of the phantom 300 when it is disposed in additional positions.

The calibration may be performed by determining the position of each imaging device of the optical surface tracking system with respect to the phantom 300. This may be performed by generating images of the phantom 300 using the imaging devices, and determining the locations and/or relative separations of one or more of the features of the phantom 300 in the image(s).

Based on the known position of the phantom 300 with respect to the coordinate system of the radiotherapy device 100 and the determined position of the phantom 300 with respect to the optical surface tracking system, the position of the optical surface tracking system with respect to the coordinate system of the radiotherapy device 100 may be determined. In other words, the three-dimensional spatial coordinates and the three-dimensional orientation of the optical surface tracking system (i.e. each imaging device thereof) may be determined in/with respect to the coordinate system of the radiotherapy device 100. In this manner, each imaging device may be calibrated with respect to the radiotherapy device 100 and also thereby calibrated with respect to each other imaging device.

As described herein, the phantom 300 comprises the first three-dimensional structures 302 and the second three-dimensional structures 304. The provision of three-dimensional structures as opposed to two-dimensional structures may be beneficial for the calibration of the optical surface tracking system for several reasons.

An optical surface tracking system as described herein may comprise a plurality of imaging devices in different locations. It is beneficial if the imaged structure (i.e. the one or more second three-dimensional structures 302) is visible to multiple or all of the imaging devices simultaneously. This is more achievable using a three-dimensional structure than a two-dimensional structure because it is more likely to be visible from more locations. In order for the imaged structure(s) to be visible to all of the imaging devices, multiple imaged structures may be provided as part of the phantom 300 or multiple phantoms 300 may be provided. Alternatively, or in addition, the phantom 300 may be moved to different positions to be visible to further imaging devices. This may be performed by moving the patient positioning surface 104 with the phantom 300 disposed thereon or moving the phantom 300 to dispose it at different points on the patient positioning surface 104.

In addition, kV imaging is better able to identify the locations of three-dimensional structures than two-dimensional structures. As such, the locations of the first three-dimensional structures 302 may be more accurately determined by virtue of these structures being three-dimensional. This in turn enables more accurate characterisation of the optical surface tracking system.

In addition, there may be further benefits to the second three-dimensional structures 304 being three-dimensional depending on the imaging modality used to determine their location. In some examples, time-of-flight imaging may be used as the optical imaging modality. Time-of-flight cameras typically have a fairly low spatial resolution relative to that of other digital cameras, which may be addressed by making the second three-dimensional structures 304 relatively large. However, time-of-flight cameras are capable of performing depth measurements. Therefore, imaging of three-dimensional structures may enable further information regarding the locations, separations and/or orientations of the structures to be determined from the optical images.

In some alternatives, the first three-dimensional structures 302 may be replaced with one or more other features detectable by the kV imaging system, such as a checkerboard pattern comprising squares or diamonds with different (e.g. alternating) radiopacities.

In some alternatives, the first three-dimensional structures 302 may be disposed internal to another structure of the phantom and may as such may not be visible optically. For example, the first three-dimensional structures may be surrounded by/suspended in/supported by another (radio-translucent) material.

As described herein, the phantom 300 may comprise one or more first three-dimensional structures 302 and one or more second three-dimensional structures 304. Provision of multiple of these structures may enable the position of the phantom 300 to be determined, and therefore the calibration to be performed, in all six degrees of freedom. However, multiple of these structures may not be provided if for example the phantom 300 is moved to different locations, e.g. by moving the patient positioning surface 104 with the phantom 300 disposed thereon. For example, the patient positioning surface 104 could be moved through an automated sequence with kV images and optical images of the phantom 300 being generated at each of a plurality of locations of the patient positioning surface 104/of the phantom 300 during the automated sequence. This may enable the position of the phantom 300 to be determined, and therefore the calibration to be performed, in all six degrees of freedom.

While evaluation of the optical surface tracking system has been discussed in most detail herein, the described features and techniques may alternatively or additionally be used to evaluate the kV imaging system. The phantom enables evaluation of the kV imaging system, and/or the optical surface tracking system, both independently and in relation to each other. For example, the features and techniques of the present disclosure may be used to evaluate the accuracy of or calibrate the kV imaging system based on a known location of one or more of the first three-dimensional structures (e.g. fixed at or relative to the isocentre). This may further be based on a known/measured size of one or more of the first three-dimensional structures, or on a known/measured separation between two or more of the first three-dimensional structures or between one of the first three-dimensional structures and another part of the phantom.

While the methods disclosed herein are presented in a certain sequential order, this should not be taken to limit the methods to the orders presented. One or more of the method steps may be omitted or rearranged. The various steps may be performed in different orders. Various steps may be performed at the same time or substantially the same time. Herein, references to events occurring substantially at the same time may refer to events at least partially overlapping in time and/or events occurring at the same time within measurement uncertainties.

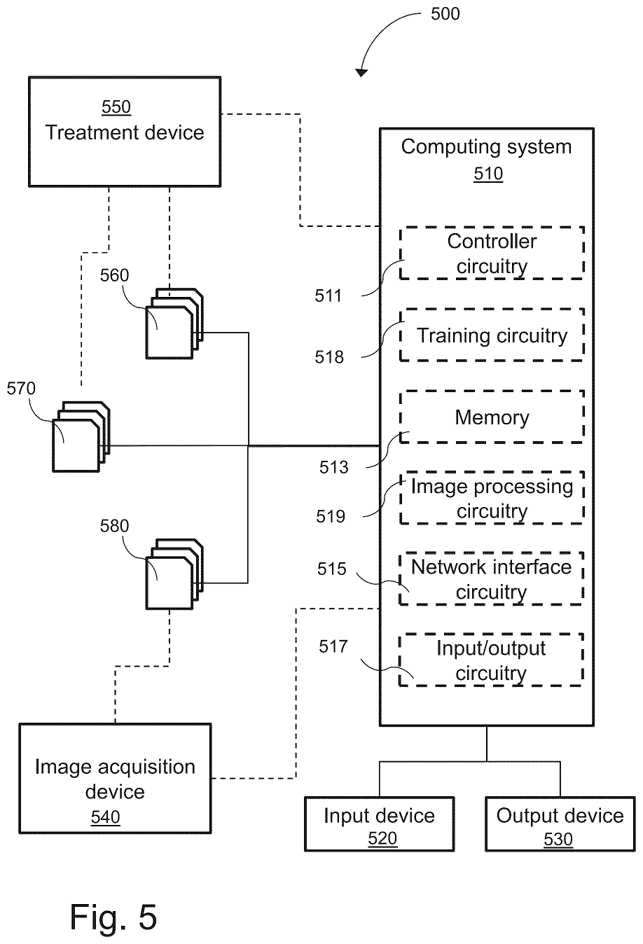

FIG. 5 illustrates a block diagram of one implementation of a radiotherapy system 500. The radiotherapy system 500 comprises a computing system 510 within which a set of instructions, for causing the computing system 510 to perform any one or more of the methods discussed herein, may be executed. The computing system 510 may also be referred to as a control device as used herein.

The computing system 510 shall be taken to include any number or collection of machines, e.g. computing device(s), that individually or jointly execute a set (or multiple sets) of instructions to perform any one or more of the methods discussed herein. That is, hardware and/or software may be provided in a single computing device, or distributed across a plurality of computing devices in the computing system. In some implementations, one or more elements of the computing system may be connected (e.g., networked) to other machines, for example in a Local Area Network (LAN), an intranet, an extranet, or the Internet. One or more elements of the computing system may operate in the capacity of a server or a client machine in a client-server network environment, or as a peer machine in a peer-to-peer (or distributed) network environment. One or more elements of the computing system may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine.

The computing system 510 includes controller circuitry 511 and a memory 513 (e.g., read-only memory (ROM), flash memory, dynamic random access memory (DRAM) such as synchronous DRAM (SDRAM) or

Rambus DRAM (RDRAM), etc.). The memory 513 may comprise a static memory (e.g., flash memory, static random access memory (SRAM), etc.), and/or a secondary memory (e.g., a data storage device), which communicate with each other via a bus (not shown).

Controller circuitry 511 represents one or more general-purpose processors such as a microprocessor, central processing unit, accelerated processing units, or the like. More particularly, the controller circuitry 511 may comprise a complex instruction set computing (CISC) microprocessor, reduced instruction set computing (RISC) microprocessor, very long instruction word (VLIW) microprocessor, processor implementing other instruction sets, or processors implementing a combination of instruction sets. Controller circuitry 511 may also include one or more special-purpose processing devices such as an application specific integrated circuit (ASIC), a field programmable gate array (FPGA), a digital signal processor (DSP), network processor, or the like. One or more processors of the controller circuitry may have a multicore design. Controller circuitry 511 is configured to execute the processing logic for performing the operations and steps discussed herein.

The computing system 510 may further include a network interface circuitry 515. The computing system 510 may be communicatively coupled to an input device 520 and/or an output device 530, via input/output circuitry 517. In some implementations, the input device 520 and/or the output device 530 may be elements of the computing system 510. The input device 520 may include an alphanumeric input device (e.g., a keyboard or touchscreen), a cursor control device (e.g., a mouse or touchscreen), an audio device such as a microphone, and/or a haptic input device. The output device 530 may include an audio device such as a speaker, a video display unit (e.g., a liquid crystal display (LCD) or a cathode ray tube (CRT)), and/or a haptic output device. In some implementations, the input device 520 and the output device 530 may be provided as a single device, or as separate devices.

In some implementations, computing system 510 includes training circuitry 518. The training circuitry 518 is configured to train a method of evaluating images and/or a method of detecting large deformations in images. For example, training circuitry 518 may train a model for performing a method of evaluating images/a method of detecting large deformations in images. The model may comprise a deep neural network (DNN), such as a convolutional neural network (CNN) and/or recurrent neural network (RNN). Training circuitry 518 may be configured to execute instructions to train a model that can be used to evaluate images/detect large deformations in images, as described herein. Training circuitry 518 may be configured to access training data and/or testing data from memory 513 or from a remote data source, for example via network interface circuitry 515. In some examples, training data and/or testing data may be obtained from an external component, such as image acquisition device 540 and/or treatment device 550. In some implementations, training circuitry 518 may be used to update, verify and/or maintain a model for determining a difference between a representation of a phantom and a reference contour of the phantom as described herein.

In some implementations, the computing system 510 may comprise image processing circuitry 519. Image processing circuitry 519 may be configured to process image data 580 (e.g. images, or imaging data), such as medical images obtained from one or more imaging data sources, a treatment device 550 and/or an image acquisition device 540 as described herein. Image processing circuitry 519 may be configured to process, or pre-process, image data. For example, image processing circuitry 519 may convert received image data into a particular format, size, resolution or the like. In some implementations, image processing circuitry 519 may be combined with controller circuitry 511.

In some implementations, the radiotherapy system 500 may further comprise an image acquisition device 540 and/or a treatment device 550, such as those disclosed herein (e.g. in relation to FIG. 1a and FIG. 1b). The image acquisition device 540 and the treatment device 550 may be provided as a single device. In some implementations, treatment device 550 is configured to perform imaging, for example in addition to providing treatment and/or during treatment. The treatment device 550 comprises the main radiation delivery components of the radiotherapy system described herein.

Image acquisition device 540 may be configured to perform positron emission tomography (PET), computed tomography (CT), magnetic resonance imaging (MRI), etc.

Image acquisition device 540 may be configured to output image data 580, which may be accessed by computing system 510. Treatment device 550 may be configured to output treatment data 560, which may be accessed by computing system 510.

Computing system 510 may be configured to access or obtain treatment data 560, planning data 570 and/or image data 580. Treatment data 560 may be obtained from an internal data source (e.g. from memory 513) or from an external data source, such as treatment device 550 or an external database. Planning data 570 may be obtained from memory 513 and/or from an external source, such as a planning database. Planning data 570 may comprise information obtained from one or more of the image acquisition device 540 and the treatment device 550.

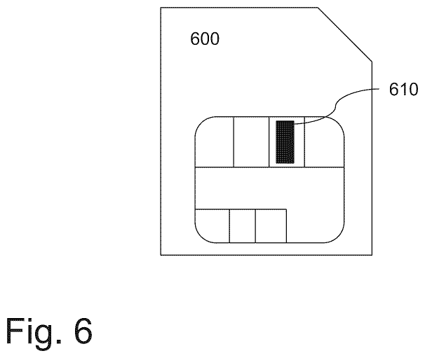

The various methods described above may be implemented by a computer program. The computer program may include computer code (e.g. instructions) 610 arranged to instruct a computer to perform the functions of one or more of the various methods described above. The steps of the methods described above may be performed in any suitable order. The computer program and/or the code 610 for performing such methods may be provided to an apparatus, such as a computer, on one or more computer readable media or, more generally, a computer program product 600)), depicted in FIG. 6. The computer readable media may be transitory or non-transitory. The one or more computer readable media 600 could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD. The instructions 610 may also reside, completely or at least partially, within the memory 513 and/or within the controller circuitry 511 during execution thereof by the computing system 510, the memory 513 and the controller circuitry 511 also constituting computer-readable storage media.]

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may comprise a special-purpose processor, such as an FPGA or an ASIC. A hardware component may also include programmable logic or circuitry that is temporarily configured by software to perform certain operations.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying,” “obtaining,” “detecting,” “generating,” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A phantom configured for evaluating imaging for radiotherapy, the phantom comprising:
a first three-dimensional structure configured for detection by a kV imaging system; and
a second three-dimensional structure configured for detection by an optical surface tracking system and spaced apart from the first three-dimensional structure.

2. The phantom according to claim 1, wherein at least one of the first three-dimensional structure or the second three-dimensional structure is spherical.

3. The phantom according to claim 1, wherein the first three-dimensional structure is formed of a ceramic.

4. The phantom according to claim 1, wherein a surface of the second three-dimensional structure is lighter in color than other surfaces of the phantom.

5. The phantom according to claim 1, wherein the second three-dimensional structure is larger in volume than the first three-dimensional structure.

6. The phantom according to claim 1, wherein the phantom comprises a support frame, and wherein the first three-dimensional structure and the second three-dimensional structure are fixedly connected to the support frame.

7. The phantom according to claim 6, wherein at least one of: i) the first three-dimensional structure is fixedly connected to the support frame via a support column or ii) wherein the second three-dimensional structure is connected to the support frame via the support column.

8. The phantom according to claim 1, further comprising:
a plurality of first three-dimensional structures configured for detection by the kV imaging system, wherein the plurality of first three-dimensional structures are spaced apart from each other.

9. The phantom according to claim 1, further comprising:
a plurality of second three-dimensional structures configured for detection by the optical surface tracking system, wherein the plurality of second three-dimensional structures are spaced apart from each other.

10. The phantom according to claim 1, further comprising:
one or more visual markings on an exterior surface thereof for laser-aided positioning of the phantom.

11. A radiotherapy device comprising:
a radiation source;
a patient positioning surface;
a kV imaging system;
an optical surface tracking system; and
a phantom disposed on the patient positioning surface, the phantom comprising:
a first three-dimensional structure configured for detection by a kV imaging system; and
a second three-dimensional structure configured for detection by an optical surface tracking system and spaced apart from the first three-dimensional structure.

12. The radiotherapy device according to claim 11, wherein the phantom is disposed at an isocenter of the radiotherapy device.

13. The radiotherapy device according to claim 11, wherein the kV imaging system is configured to generate a kV image of a kV imaging volume, wherein the first three-dimensional structure is disposed within the kV imaging volume, and wherein the second three-dimensional structure is disposed outside of the kV imaging volume.

14. The radiotherapy device according to claim 11, wherein the optical surface tracking system is a time-of-flight imaging system.

15. A method for evaluating imaging for radiotherapy, the method comprising:
obtaining one or more spatial measurements of a phantom, wherein the phantom comprises:
a first three-dimensional structure configured for detection by a kV imaging system; and
a second three-dimensional structure configured for detection by an optical surface tracking system and spaced apart from the first three-dimensional structure;
generating, using a kV imaging system, a kV image of the phantom;
generating, using an optical surface tracking system, an optical image of the phantom; and
comparing the one or more spatial measurements to the kV image and the optical image.

16. The method according to claim 15, further comprising:
determining, based on the comparing and based on a predetermined error margin for the kV imaging system; and
an error margin for the optical surface tracking system.

17. The method according to claim 15, wherein at least one of: i) obtaining the one or more spatial measurements comprises determining a center of the first three-dimensional structure and the comparing comprises comparing the kV image to the determined center, or ii) the obtaining the one or more spatial measurements comprises determining a center of the second three-dimensional structure and the comparing comprises comparing the optical image to the determined center.

18. The method according to claim 15, further comprising:
generating the kV image and the optical image with the phantom disposed on a patient positioning surface of a radiotherapy device.

19. The method according to claim 18, further comprising:
moving the patient positioning surface to at least one of an adjusted location or an adjusted orientation with the phantom disposed thereon;
generating, using the kV imaging system, a second kV image of the phantom;
generating, using the optical surface tracking system, a second optical image of the phantom; and
comparing the spatial measurements to the second kV image and the second optical image.

20. The method according to claim 15, further comprising:
calibrating one or more imaging devices of the optical surface tracking system based on the comparing.
